(19) United States
(12) Patent Application Publication
(10) Pub. No.: US 2024/0392354 A1
(43) Pub. Date: Nov. 28, 2024

(54) Multimode Omics of Single Tissue Sample

(71) Applicants: CZ BIOHUB SF, LLC, San Francisco, CA (US); THE REGENTS OF THE UNIVERSITY OF CALIFORNIA, Oakland, CA (US)

(72) Inventors: Adam Abate, Oakland, CA (US); Linfeng Xu, Oakland, CA (US)

(21) Appl. No.: 18/690,840

(22) PCT Filed: Sep. 15, 2022
(86) PCT No.: PCT/US22/43624

§ 371 Date: Mar. 11, 2024

Related U.S. Application Data
(60) Provisional application No. 63/245,371, filed on Sep. 17, 2021.

(51) Int. Cl. C12Q 1/6841; C12Q 1/6816; G01N 21/64; G01N 21/65; G01N 33/68

(52) U.S. Cl. CPC C12Q 1/6841; C12Q 1/6816; G01N 21/64; G01N 21/65; G01N 33/6848; G01N 2021/653

(57) ABSTRACT

The present disclosure provides materials and methods for obtaining multiple measurements from the same sample. For example, the present disclosure provides methods to perform spatial metabolomics (using, for example, mass spectrometry imaging), transcriptomics and fluorescent imaging of the same tissue section.

### CROSS-REFERENCE TO RELATED APPLICATIONS

The present application claims priority to U.S. Provisional Patent Application No. 63/245,371 filed Sep. 17, 2021 the entirety of which is incorporated by reference herein.

### FIELD

The present disclosure relates generally to methods for multimode omics of tissue sections using, for example, mass spectrometry imaging and spacial transcriptomics and genomics.

### BACKGROUND

Currently, mass spec imaging based on MALDI MS is the best way to show the distribution of small chemicals or metabolites of tissue (e.g., brain tissue). However, the current techniques require the substrate to be conductive and thus it cannot be combined with commercially available gene expression techniques and materials (e.g., slides). Measurement of gene expression levels of brain tissues generally requires florescent probes to stain the tissue in order to link mRNA data with other information based on the microscope imaging. However, there are no proper probes to label small chemicals or metabolites, and thus there are currently no methods available to link metabolomics and transcriptomics or genomics with spatial control. Thus, there remains a need for a method of obtaining such linked information.

### SUMMARY OF THE INVENTION

One embodiment of the present disclosure provides a method obtaining multiple measurements from one tissue sample, said method comprising the steps of: (a) preparing a tissue sample, wherein said preparing comprises placing the tissue sample on a substrate capable of labeling the tissue sample with spatial barcodes; (b) obtaining a first measurement from the tissue sample, wherein said first measurement does not substantially disrupt the integrity of the tissue sample; (c) obtaining a last measurement from the tissue sample, wherein said last measurement comprises obtaining spatial information for one or more biomolecules in the tissue sample; thereby obtaining multiple measurements from one tissue sample.

In another embodiment, the method is provided further comprising obtaining one or more additional measurements after the first measurement and before the last measurement, wherein the additional measurements do not substantially disrupt the integrity of the tissue sample. In still another embodiment, 1, 2, 3, 4, 5 or more additional measurements are obtained before the last measurement. In another embodiment, the first measurement and the additional measurements are obtained using the same or different techniques. In another embodiment, the first measurement and the 1, 2, 3, 4, 5 or more additional measurements are carried out in in any order.

In still other embodiment, an aforementioned method is provided wherein the spatial information obtained by the last measurement is linked to the first measurement and/or the one or more additional measurements. In still other embodiments, the tissue sample in (a) is sectioned prior to labeling with spatial barcodes. In another embodiment, the tissue sample is sectioned by a method selected from the group consisting of cryosectioning, formalin fixation, laser cutting and laser capture microdissection (LCM).

The present disclosure also provides an aforementioned method wherein the spatial barcodes comprise nucleic acid barcodes. In other embodiments, the one or more biomolecules is selected from the group consisting of a nucleic acid, a lipid, a small molecule, a sugar, a protein, a nuclei, and a cell. In still other embodiments, the tissue sample is about of 0.1 to 1,000 micron thickness. In another embodiment, the tissue sample is a human or animal biopsy. In another embodiment, the biopsy comprises a sample from a tumor, lymphatic tissue, infected tissue, immune infiltrated tissue, central nervous system tissue, digestive system tissue, developing tissue; whole animal section, and microbial community.

In yet another embodiment, an aforementioned method is provided wherein the first measurement, and optionally the one or more additional measurements, is obtained using a technique selected from the group consisting of optical measurement, spectrographic measurement, and a microscopic measurement. In another embodiment, the optical measurement is a microscopic measurement selected from the group consisting of wide field, epi-fluorescent, coherent Raman scattering (CRS), confocal, total internal reflectance fluorescence (TIRF), and structured illumination microscopy (SIM). In another embodiment, the spectrographic measurement is selected from the group consisting of desorption electrospray ionization mass spectrometry (DESI MS), matrix-assisted laser desorption/ionization mass spectrometry (MALDI MS), matrix assisted laser desorption/ionization-time of flight mass spectrometry (MALDI-TOF MS), and secondary ion mass spectrometry (SIMS). In another embodiment, prior to obtaining the first measurement with MALDI MS or MALDI-TOF MS, the tissue sample in (a) is further prepared comprising labeling with a conductive membrane. In still another embodiment, the conductive membrane (e.g., materials that have a surface resistance of less than 1×105 ohms/square like metals) is an indium tin oxide (ITO) membrane. In yet another embodiment, the microscopic measurement is selected from the group consisting of atomic force microscopy (AFM) and near-field scanning optical microscopy (NSOM).

The present disclosure also provides an aforementioned method wherein the last measurement comprises obtaining sequence and location information for one or more nucleic acids in the tissue sample. In another embodiment, the last measurement comprises obtaining spatial transcriptomic information, or spatial genomic information. In another embodiment, said obtaining comprises (i) lysing cells of the tissue sample under conditions that release mRNA from the lysed cells; (ii) capturing the mRNA with corresponding nucleic acid barcodes; and (iii) determining the sequence of captured the mRNA. In another embodiment, said determining the sequence of captured mRNA comprises reverse transcribing the mRNA into cDNA, amplifying the cDNA using polymerase chase reaction (PCR), and sequencing the amplified cDNA.

In some embodiments, an aforementioned method is provided that links spatial information of one or more biomolecules, and, e.g., a metabolite measurement, to the same location or same cell or cell type.

The present disclosure provides in one embodiment a method of obtaining a mass spectrographic measurement and spatial transcriptomic information from the same tissue section sample, said method comprising the steps of: (a) preparing a tissue sample of about 0.1 to 1,000 micron thickness, wherein said preparing comprises (i) cryosectioning the tissue sample; (ii) placing the tissue sample on a slide that has been pre-coated with nucleic acid barcodes capable of labeling nucleic acids within the tissue sample; and (iii) placing a conductive membrane on the tissue sample; (b) obtaining at least one mass spectrographic measurement from the tissue sample using MALDI MS, wherein said measurement does not substantially disrupt the integrity of the tissue sample; (c) obtaining spatial transcriptomic information from the tissue sample, wherein said obtaining comprises (i) lysing cells of the tissue sample under conditions that release mRNA from the lysed cells; (ii) capturing the mRNA with corresponding nucleic acid barcodes; and (iii) determining the sequence of captured the mRNA; and optionally (d) linking the at least one mass spectrographic measurement of (b) with the spatial transcriptomic information of (c); thereby obtaining a mass spectrographic measurement and spatial transcriptomic information from the same tissue section sample.

### DETAILED DESCRIPTION

The present disclosure provides, in various embodiments described herein, methods to meet the aforementioned need in the art by performing, for example, both mass spectrometry imaging and mRNA sequencing techniques of the same tissue sample. By way of example, the present disclosure provides in various embodiments methods to perform spatial metabolomics (using, for example, mass spectrometry imaging), transcriptomics (using, for example, a Visium slide from 10× Genomics) and fluorescent imaging of the same tissue section (for example, a brain tissue section). In this way, the present disclosure enables the linking of multiple types and points of data—for example data from mass spectrometry imaging with mRNA (e.g., identification of cell types based on mRNA sequences and metabolites from the same type of cells). As described herein, the present disclosure provides methods that use a conductive membrane placed on top of the tissue to perform the mass spectrometry imaging.

As described herein, in one embodiment of the present disclosure provides a method of for generating multimode omics information of a tissue section with spatial control. In some embodiments, the methods described herein comprise one or more of the following steps:
1. Preparing a tissue section of 0.1 to 1000 micron thickness;
2. Perform nondestructive measurement;
3. Perform partial destructive measurement;
4. Where additional versions of steps 2 and 3 can be performed with similar or different measurements in additional steps; and
5. Where spatial information gathered from at least one of the steps is used to link at least two of the measurements.

Thus, in some embodiments, the present disclosure provides methods that include, for example, preparing tissue section on a suitable slide (e.g., a slide coated with probe and/or barcodes to capture nucleic acids), usually by cryosectioning (0.1 to 1000 micron, or 10-30 more typical (determines resolution of z axis)); performing one or more nondestructive measurements, like AFM, optical imaging or after staining; performing one or more relatively moderately, partially destructive (e.g., usually only involving the top surface of the tissue section) measurement of the tissue section, for example mass spec imaging using MALDI or DESI; performing one or more relatively destructive (e.g., usually requiring lysis of cells and sequencing) measurement of the whole tissue section, for example spatial transcriptomics, genomics or epigenomics; and linking (e.g., assessing different pieces of data or information obtained from the aforementioned steps) at least two of the measurements according to the spatial information of the tissue section.

As used herein, the term “sample” or “biological sample” or “tissue sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, serum, plasma, biological fluid, and tissue samples. “Sample” and “biological sample” includes cells, e.g., bacterial cells or eukaryotic cells; biological fluids such as blood, cerebrospinal fluid, semen, saliva, and the like; bile; bone marrow; skin (e.g., skin biopsy); and viruses or viral particles obtained from an individual.

As described more fully herein, in various aspects the subject methods may be used to detect a variety of components from such biological samples. Components of interest include, but are not necessarily limited to, cells (e.g., circulating cells and/or circulating tumor cells), viruses, polynucleotides (e.g., DNA and/or RNA), polypeptides (e.g., peptides and/or proteins), and many other components that may be present in a biological sample.

The terms “polynucleotide” and “nucleic acid” and “target nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA. In particular, the polynucleotides and nucleic acids, is used herein to refer to a binding moiety used in the methods described herein and/or as a target of the methods described herein (e.g., a target whose location and sequence is determined by practicing the methods described herein).

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, CA)) according to specifications provided by the manufacturer or they can be the result of restriction enzyme digestion and fractionation.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single-stranded or double-stranded.

The term “nucleic acid array” as used herein refers to a regular organization or grouping of nucleic acids of different sequences immobilized on a solid phase support at known locations. The nucleic acid can be an oligonucleotide, a polynucleotide, DNA, or RNA. The solid phase support can be silica, a polymeric material, glass, beads, chips, slides, or a membrane. The methods of the present invention are useful with both macro-and micro-arrays.

The term “protein” or “protein of interest” (e.g., as it relates to a target biomolecule) refers to a polymer of amino acid residues, wherein a protein may be a single molecule or may be a multi-molecular complex. The term, as used herein, can refer to a subunit in a multi-molecular complex, polypeptides, peptides, oligopeptides, of any size, structure, or function. It is generally understood that a peptide can be 2 to 100 amino acids in length, whereas a polypeptide can be more than 100 amino acids in length. A protein may also be a fragment of a naturally occurring protein or peptide. The term protein may also apply to amino acid polymers in which one or more amino acid residues is an artificial chemical analogue of a corresponding naturally occurring amino acid. A protein can be wild-type, recombinant, naturally occurring, or synthetic and may constitute all or part of a naturally-occurring, or non-naturally occurring polypeptide. The subunits and the protein of the protein complex can be the same or different. A protein can also be functional or non-functional.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al, Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

“Detecting” or “determining” or “measuring” as used herein generally means identifying the presence of a target, such as a target nucleic acid or protein or biomolecule. In various embodiments, detection signals are produced by the methods described herein, and such detection signals may be optical signals which may include but are not limited to, colorimetric changes, fluorescence, turbidity, and luminescence. Detecting, in still other embodiments, also means quantifying a detection signal, and the quantifiable signal may include, but is not limited to, transcript number, amplicon number, protein number, and number of metabolic molecules. In this way, sequencing or bioanalyzers are employed in certain embodiments.

According to some embodiments of the present disclosure, a lysing reagent is used in the methods. Lysing agents may include, for example chemical lysis, such as SDS, detergents, alkaline, and acid; biological lysis, such as lysis enzymes, viruses, and phages; and physical lysis such as beads beating, grinding, frozen-thaw, and sonication, heating, cutting, and laser or ion beams.

The present disclosure provides methods of detecting a target in a sample, where the target may be, for example, a nucleic acid (RNA, DNA), biomolecules such nucleic acids, genes, proteins or polypeptides or epitopes, as well as biological particles such as cells (bacterial, human, parasite) and viruses.

As used herein, “biomolecules” can be nucleic acids themselves or can be other biomolecules that are associated with nucleic acids or comprise nucleic acids such as cells, proteins, or nuclei and the like. The term “substrate” or label substrate” or “labelling substrate” includes, without limitation, a slide or an array or, in one embodiment can be the tissue sample itself.

As described herein, the term “non-destructive” measurement means a measurement technique that does not substantially disrupt the integrity of the sample being measured (e.g., a tissue sample). Exemplary non-destructive measurements are described herein and include, in various embodiments, optical measurement, spectrographic measurement, and a microscopic measurement.

As used herein, the term “partially destructive” measurement means a measurement technique that may partially or moderately disrupt the integrity of the sample being measured (e.g., a tissue sample). Exemplary partially destructive measurements are described herein and include, in various embodiments, mass spec imaging using MALDI or DESI.

Partially destructive measurement techniques may compromise all or a portion of the sample. In some embodiments of the present disclosure, a method step, usually the last or near-last method step, may be more destructive than the above-mentioned techniques (e.g., destructive or partially destructive) whereby the integrity of the sample is disrupted. Examples of such techniques are described herein and include, in various embodiments, sequencing, spatial transcriptomic or spatial genomic methods that require cell lysis.

In one exemplary embodiment of the present disclosure, multiple, partially destructive measurements are performed on a sample. For example, mass spec ion imaging followed by spatial transcriptomics may be performed as follows:

### Mass Spec Ion Imaging

1. For DESI MS imaging: No special requirement for sample preparation to perform DESI MS imaging;
2. For MALDI MS imaging:
   a. Add a conductive membrane on top of the tissue section;
   b. Add solvent (methanol) conductive membrane (˜10 microns thick), wait a few minutes to dry; and
   c. Perform MS to top of membrane.

For spatial transcriptomics: Label a substrate slide or the tissue section with spatial barcode, for example a DNA barcode.
1. Lysis cells to release RNA;
2. Capture RNA with corresponding spatial barcodes from above;
3. Perform reverse transcription to get the cDNA integrated with spatial barcodes;
4. Perform PCR to amplify the cDNA and prepare it for sequencing; and
5. Sequencing to get the transcriptomics information and link it to the spatial information based on the spatial barcodes.

In other embodiments, for example for spatial genomics and spatial epigenomics, the steps are similar while instead of RNA, genome DNA or epigenome DNA are captured with corresponding spatial barcodes.

As described herein, the present disclosure provides various methods that allow multimodal analysis of tissue sections with spatial resolution. The present disclosure provides methods and steps to perform the different modes of analysis in a sequence that permits later modalities to be performed, for instance, beginning with less destructive measurements, and performing additional (increasingly) more destructive measurements with subsequent steps. Multiple modes can be applied in each step, for example, applying multiple less destructive methods such as imaging, spectroscopy, AFM, etc, in a sequence. Moreover these steps can be performed in different orders and multiple steps can be added and repeated in any sequence that is optimal for the desired multi-modal analysis.

### Tissue Section Preparation

The present disclosure provides various methods useful for analyzing tissue sections comprising largely 2D features, with a thickness ranging from 0.1 to 1000 microns. The disclosure provides methods for repeated measurements on such a tissue section preserving integrity to allow multiple follow-on measurements. The methods provided herein are initiated, in some embodiments, by first preparing a tissue section, for example by cryosectioning or formalin fixation and sectioning. The sectioned tissue is then placed on a substrate. In some embodiments, the substrate is a slide engineered with a mechanism for positionally barcoding nucleic acids that forms the tissue section to, for example, obtain spatially resolved genomics information.

In some embodiments, a tissue section of 0.1 to 1000 micron thickness is prepared for example by freezing and/or slicing the tissue into layers. The tissue can be processed as is, or subjected to additional processing to facilitate analysis. For example, the tissue can be perfused with matrix molecules that facilitate gel expansion, to enable high resolution microscopy or barcoding, for highly spatially resolved single cell sequencing. Tissues may also be fixed or sliced fresh. Components zipfrom the tissues may be captured onto the perfused matrix, such as nucleic acids, lipids, small molecules, sugars, proteins, etc.

They may also be subjected to further processing, for example, to label with probes, such as antibodies or fluorescent oligonucleotides. Tissue sections may be printed with components, such as oligonucleotides, dyes, and beads, to facilitate downstream analysis. For example, in one embodiment, printing of randomly labeled beads can be used to spatially label tissue sections based on a broadcasting triangulation. Tissue components can be subjected to chemical or enzymatic processing to facilitate additional analysis, such as to enable methylation or other epigenetic sequencing. For example, tagmentation can facilitate amplification of sequences for library preparation, or to perform ATAC seq analysis. Enzymes and other components can facilitate lysis, such as cell wall digesting agents when applied to certain cell types, such as fungi or microbes, or proteases to facilitate digestion of cell components, such as chromatin. Tissues may be processed by printing materials on them using available modalities, such as spatial “zipcodes,” dyes, chemicals, etc. using inkjet, laserjet, and dispensing technologies.

These steps can be done on fixed, fresh, frozen, gelled, or expanded tissue sections. For example, in one embodiment, to facilitate highly resolved single cell methylation DNA sequencing, tissues can be perfused with gel matrix, expanded, and processed with bisulfate. The prepared tissues can be analyzed, before, during, or after processing. For example, imaging spectroscopy and mass spectrometry can be used to obtain information about the sample in its spatial context. The sample may be subjected to fluorescence in situ hybridization analysis (FISH) to generate FISH based images that can facilitate spatial sequencing of the tissues by combining FISH with other forms of sequence analysis, such as barcode based sequencing. This can be accomplished, for example, by matching FISH patterns with patterns identified in the barcode sequencing data.

### Nondestructive Measurements

After or before the tissue section is prepared and mated to the slide, multiple measurements can be performed that have differing levels of destructiveness. In this way, the order the measurements are performed in sequence will preserve the tissue integrity to enable follow-on measurements. For example in general, one would not perform a wholly destructive measurement at the initiation of the sequence, because this would negate the ability to perform additional measurements. For example, in general, one would not perform single cell RNAseq requiring complete lysis of the tissue at the first step, since afterwards it would no longer be possible to image the tissue section and obtain imaging data. Rather, in one embodiment, one would first perform imaging, then RNAseq, in this example. Moreover, some modalities are more destructive than others, and a method need not be completely nondestructive to precede follow on measurements, provided the tissue section retains sufficient integrity for the follow on measurements. For Example, MALDI mass spec imaging of the tissue section is moderately destructive since it ablates portions of the tissue section that may no longer yield molecules for sequencing. However, other portions of the same tissue section may still provide the necessary molecules. Alternatively, the MALDI measurements can be configured to specifically target certain molecules, and thereby preserving others. For example based on matrix choices and laser power and other properties that will be apparent to those of ordinary skill in the art, small molecules may be ablated and used for analysis in the mass spec, but macromolecules such as nucleic acids, may not and thus be retained in the substrate. Under such a circumstance, the MALDI is partially destructive of the tissue section, but much of the material useful for the follow-on measurements is retained and thus allows effective single cell sequencing.

Optical, spectrographic, and AFM modalities are generally nondestructive and do not significantly alter tissue section properties. Thus, these modalities can generally be performed at any point in the sequence, and can optionally be performed repeatedly. For example, a section can be optically imaged, analyzed with MALDI mass spec, and imaged again. Performing the imaging repeatedly may improve data quality and provide useful information about how the MALDI perturbed the sample, to better accumulate or interpret follow on measurements. When selecting the order of measurements, it is important to recognize how each measurement perturbs the sample so as to properly sequence the measurements. For example, when performing the optical+MALDI+optical measurement described above, the MALDI may require addition of a matrix that may interfere with the second optical measurement. If AFM is to be performed, it is important that this occurs before application of the matrix, which could interfere with that measurement.

### Partially Destructive Measurements

As described herein, some measurements may be more or less destructive than others. For example, sequencing generally requires that nucleic acids be extracted from the tissue section and barcoded to achieve spatial information. In such a case, tissue lysis may be required to free mRNA or genomic DNA from the tissue's cells. Such lysis procedures are partially or, in some cases, wholly destructive of the tissue section and thus, generally, should be performed as one of the last steps in the measurement sequence.

### Linking Spatial Information With Additional Measurements

As described herein, the methods of the present disclosure enable multiple and optionally different measurements in a multimodal analysis in such a way that spatial information can be linked together. For example, optical imaging data is often obtained in the form of a picture comprising a two dimensional array of pixel values corresponding to coordinates of the tissue section. When run in “imaging” mode, MALDI and/or DESI mass spectrometry can be used to obtain a similar said image, but in this case the pixel data comprises a high dimensional vector of the mass spectrum for different m/z values. In some aspects, the MALDI image is analogous to the optical image, where the different m/z of the spectra correspond to different “color” values, and in which the “pixels” likewise correspond to positions of the tissue section. In this way, optical intensity values that, for instance, may represent the expression of a fluorescent protein, can be related to the mass spectrum data for the same “pixel” volume in the tissue. In this way, these disparate datasets can be linked together using the methods described in this patent. If a sequence of multiple modalities is applied, then at least some of them can be spatially linked in this way. For example, if spatially resolved sequencing data is obtained, barcodes existing in the sequence data can be used to determine from which “pixel” reads originated on the substrate and, thus, used to link this data to the others. Thus, using the methods described herein, it is possible to perform a complex, multi-step sequence of nondestructive and partially destructive measurements on a tissue section, and link at least some of them based on their spatial information.

In one embodiment, a method of obtaining multiple measurements from one tissue sample is provided, said method comprising the steps of: (a) preparing a tissue sample, wherein said preparing comprises placing the tissue sample on a substrate capable of labeling the tissue sample with spatial barcodes; (b) obtaining a first measurement from the tissue sample, wherein said first measurement does not substantially disrupt the integrity of the tissue sample; (c) obtaining a last measurement from the tissue sample, wherein said last measurement comprises obtaining spatial information for one or more biomolecules in the tissue sample; thereby obtaining multiple measurements from one tissue sample. As used herein, the term “spatial barcode” refers to a barcode, for example a nucleic acid barcode as known in the art, that can be used to record or identify the location of a biomolecule within a sample, e.g., a tissue sample.

As described herein, the methods include “labelling the tissue sample” in various embodiments. As used herein, the term “labelling” refers to conjugating or otherwise attaching a label, such as a spatial barcode. In some embodiments, “spatial information” is obtained by one or more measurements of the methods provided herein. “Spatial information” means, for example, the location of a cell or cell type or collection of cells within the tissue sample.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of obtaining multiple measurements from one tissue sample, said method comprising the steps of:
(a) preparing a tissue sample, wherein said preparing comprises placing the tissue sample on a substrate capable of labeling the tissue sample with spatial barcodes;
(b) obtaining a first measurement from the tissue sample, wherein said first measurement does not substantially disrupt the integrity of the tissue sample;
(c) obtaining a last measurement from the tissue sample, wherein said last measurement comprises obtaining spatial information for one or more biomolecules in the tissue sample;
thereby obtaining multiple measurements from one tissue sample.

2. (canceled)

3. The method of claim 1, wherein 1, 2, 3, 4, 5 or more additional measurements are obtained before the last measurement, and wherein the additional measurements do not substantially disrupt the integrity of the tissue sample.

4. The method of claim 3, wherein the first measurement and the additional measurements are obtained using the same or different techniques.

5. The method of claim 3, wherein the first measurement and the 1, 2, 3, 4, 5 or more additional measurements are carried out in in any order.

6. The method of claim 1, wherein the spatial information obtained by the last measurement is linked to the first measurement and/or the one or more additional measurements.

7. The method of claim 1, wherein the tissue sample in (a) is sectioned prior to labeling with spatial barcodes.

8. The method of claim 7, wherein the tissue sample is sectioned by a method selected from the group consisting of cryosectioning, formalin fixation, laser cutting and laser capture microdissection (LCM).

9. The method of claim 1, wherein the spatial barcodes comprise nucleic acid barcodes.

10. The method of claim 1, wherein the one or more biomolecules is selected from the group consisting of a nucleic acid, a lipid, a small molecule, a sugar, a protein, a nuclei, and a cell.

11. The method of claim 1, wherein the tissue sample is about of 0.1 to 1,000 micron thickness.

12. (canceled)

13. The method of claims 11, wherein the biopsy comprises a sample from a tumor, lymphatic tissue, infected tissue, immune infiltrated tissue, central nervous system tissue, digestive system tissue, developing tissue; whole animal section, and microbial community.

14. The method of claim 1, wherein the first measurement, and optionally one or more additional measurements, is obtained using a technique selected from the group consisting of optical measurement, spectrographic measurement, and a microscopic measurement.

15. The method of claim 14, wherein the optical measurement is a microscopic measurement selected from the group consisting of wide field, epi-fluorescent, coherent Raman scattering (CRS), confocal, total internal reflectance fluorescence (TIRF), and structured illumination microscopy (SIM).

16. The method of claim 14, wherein the spectrographic measurement is selected from the group consisting of desorption electrospray ionization mass spectrometry (DESI MS), matrix-assisted laser desorption/ionization mass spectrometry (MALDI MS), matrix assisted laser desorption/ionization-time of flight mass spectrometry (MALDI-TOF MS), and secondary ion mass spectrometry (SIMS).

17. The method of claim 16, wherein prior to obtaining the first measurement with MALDI MS or MALDI-TOF MS, the tissue sample in (a) is further prepared comprising labeling with a conductive membrane.

18. The method of claim 17, wherein the conductive membrane is an indium tin oxide (ITO) membrane.

19. The method of claim 14, wherein the microscopic measurement is selected from the group consisting of atomic force microscopy (AFM) and near-field scanning optical microscopy (NSOM).

20. The method of claim 1, wherein the last measurement comprises obtaining sequence and location information for one or more nucleic acids in the tissue sample.

21. The method of claim 20, wherein the last measurement comprises obtaining spatial transcriptomic information, or spatial genomic information.

22. (canceled)

23. (canceled)

24. A method of obtaining a mass spectrographic measurement and spatial transcriptomic information from the same tissue section sample, said method comprising the steps of:
(a) preparing a tissue sample of about 0.1 to 1,000 micron thickness, wherein said preparing comprises (i) cryosectioning the tissue sample; (ii) placing the tissue sample on a slide that has been pre-coated with nucleic acid barcodes capable of labeling nucleic acids within the tissue sample; and (iii) placing a conductive membrane on the tissue sample;
(b) obtaining at least one mass spectrographic measurement from the tissue sample using MALDI MS, wherein said measurement does not substantially disrupt the integrity of the tissue sample;
(c) obtaining spatial transcriptomic information from the tissue sample, wherein said obtaining comprises (i) lysing cells of the tissue sample under conditions that release mRNA from the lysed cells; (ii) capturing the mRNA with corresponding nucleic acid barcodes; and (iii) determining the sequence of captured the mRNA; and optionally
(d) linking the at least one mass spectrographic measurement of (b) with the spatial transcriptomic information of (c);
thereby obtaining a mass spectrographic measurement and spatial transcriptomic information from the same tissue section sample.
